(19) United States
(12) Patent Application Publication
(10) Pub. No.: US 2007/0067020 A1
(43) Pub. Date: Mar. 22, 2007

(54) Intraluminal stent, delivery system, and a method of treating a vascular condition

(75) Inventor: Susan Rea, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vasular, Inc., Santa Rosa, CA (US)

(21) Appl. No.: 11/233,591

(22) Filed: Sep. 22, 2005

(51) Int. Cl. A61F 2/06

(52) U.S. Cl. 623/1.22

(57) ABSTRACT

An intraluminal stent, an intraluminal stent delivery system, and a method treating a vascular condition. The stent includes a framework including an elongated spiral seam. The framework includes a side edge including an angle formed with respect to an end edge. The system includes a catheter and a stent. The stent includes a framework including an elongated spiral seam. The framework includes a side edge including an angle formed with respect to an end edge. The method includes positioning a stent including at least one spiral seam at a target region of a vessel. The stent is expanded from a compressed configuration to a deployed configuration. At least one therapeutic agent is delivered from the stent along the at least one spiral seam.

### TECHNICAL FIELD OF THE INVENTION

The present invention relates generally to the field of implantable medical devices. More particularly, the invention relates to an intraluminal stent, delivery system, and method of treating a vascular condition.

### BACKGROUND OF THE INVENTION

Balloon angioplasty is a medical procedure to widen obstructed blood vessels narrowed by plaque deposits. The procedure may be used in coronary or peripheral arteries. In an angioplasty procedure, a catheter having a special inflatable balloon on its distal end is navigated through the patient's arteries and is advanced through the artery to be treated to position the balloon within the narrowed region (stenosis). The region of the stenosis is expanded by inflating the balloon under pressure to forcibly widen the artery. After the artery has been widened, the balloon is deflated and the catheter is removed from the patient.

A significant difficulty associated with balloon angioplasty is that in a considerable number of cases the artery may again become obstructed in the same region where the balloon angioplasty had been performed. The repeat obstruction may be immediate (abrupt reclosure), which is usually caused by an intimal flap or a segment of plaque or plaque-laden tissue that loosens or breaks free as a result of the damage done to the arterial wall during the balloon angioplasty. Such abrupt reclosure may block the artery requiring emergency surgery which, if not performed immediately, may result in a myocardial infarction and, possibly, death. This risk also necessitates the presence of a surgical team ready to perform such emergency surgery when performing balloon angioplasty procedures. More commonly, a restenosis may occur at a later time, for example, two or more months after the angioplasty for reasons not fully understood and which may require repeat balloon angioplasty or bypass surgery. When such longer term restenosis occurs, it usually is more similar to the original stenosis, that is, it is in the form of cell proliferation and renewed plaque deposition in and on the arterial wall.

To reduce the incidence of re-obstruction and restenosis, several strategies have been developed. Implantable devices, such as stents, have been used to reduce the rate of angioplasty related re-obstruction and restenosis by about half. The use of such intraluminal devices has greatly improved the prognosis of these patients. The stent is placed inside the blood vessel after the angioplasty has been performed. A catheter typically is used to deliver the stent to the arterial site to be treated. The stent may further include one or more therapeutic substance(s) impregnated or coated thereon to limit re-obstruction and/or restenosis.

Numerous stent designs are known in the art. A prior art ratchet-locking stent 100 design includes one or more, in this case one, interlocking part(s) joined at a seam 102, as shown in FIGS. 1A and 1B. The part is formed as a flat sheet and is folded upon itself to make up a tubular stent. One consideration in the design of the stent 100 relates to seam strength. Seam 102 extends from a first end of the stent 104 to a second end 106. When deployed in an artery, for example, a stent is subjected to radial force from the arterial walls. For a stent design with one or more longitudinal seams running directly along its length, localized weak points or “pinch points” may occur along the seam 102. The weak points may compromise the strength of the stent 100 and hence its ability to maintain an open lumen. As such, it would be desirable to provide a ratchet-locking stent with a relatively strong seam.

Another consideration in the design of the stent 100 relates to therapeutic agent delivery. Stents typically include one or more therapeutic agents disposed thereon. During application, therapeutic agent(s) may accumulate in and around the seam 102. For example, during the application process, the therapeutic agent(s) are commonly distributed evenly across the surface area of the stent 100. However the therapeutic agent(s) may concentrate in regions with more material, as in the seam 102 region. The accumulation may result in an uneven and overall heterogeneous drug delivery profile. As such, it would be desirable to provide a ratchet-locking stent with a more even and homogenous drug delivery profile.

Another consideration in the design of the stent 100 relates to profile size (i.e., cross-sectional diameter). It is often desirable to provide a small profile size as advancement of a device within the vasculature oftentimes includes navigating many sharp twists, turns, and narrow spaces. Relatively large devices may be more difficult to maneuver through a sometimes tortuous vasculature. Devices with smaller profiles may be less prone to contact the vascular walls during advancement and impart damage to the delicate endothelium. As such, it would be desirable to provide a stent with a relatively small profile size.

Accordingly, it would be desirable to provide an intraluminal stent, delivery system, and method of treating a vascular condition that would overcome the aforementioned and other limitations.

### SUMMARY OF THE INVENTION

A first aspect according to the invention provides an intraluminal stent. The stent includes a framework including an elongated spiral seam. The framework includes a side edge including an angle formed with respect to a frame corner.

A second aspect according to the invention provides an intraluminal stent delivery system. The stent includes a framework including an elongated spiral seam. The framework includes a side edge including an angle formed with respect to a frame corner.

A third aspect according to the invention provides a method treating a vascular condition. The method includes positioning a stent including at least one spiral seam at a target region of a vessel. The stent is expanded from a compressed configuration to a deployed configuration. At least one therapeutic agent is delivered from the stent along the at least one spiral seam.

The foregoing and other features and advantages of the invention will become further apparent from the following description of the presently preferred embodiments, read in conjunction with the accompanying drawings. The drawings have not been drawn to scale. The detailed description and drawings are merely illustrative of the invention, rather than limiting the scope of the invention being defined by the appended claims and equivalents thereof.

### BRIEF DESCRIPTION OF THE DRAWINGS

FIG. 1A is a perspective view of a prior art ratchet-locking stent;

FIG. 1B is a cross-sectional view of the prior art stent shown in FIG. 1A;

FIG. 2 is a perspective view of an intraluminal stent delivery system, in accordance with one embodiment of the present invention;

FIG. 3 illustrates a flowchart of a method of treating a vascular condition, in accordance with one embodiment of the present invention.

FIG. 4 is a perspective view of three stent units, in accordance with one embodiment of the present invention;

FIG. 5A is perspective view of an assembled stent in a deployed configuration, in accordance with the present invention; and

FIG. 5B is cross-sectional view of the stent shown in FIG. 5A.

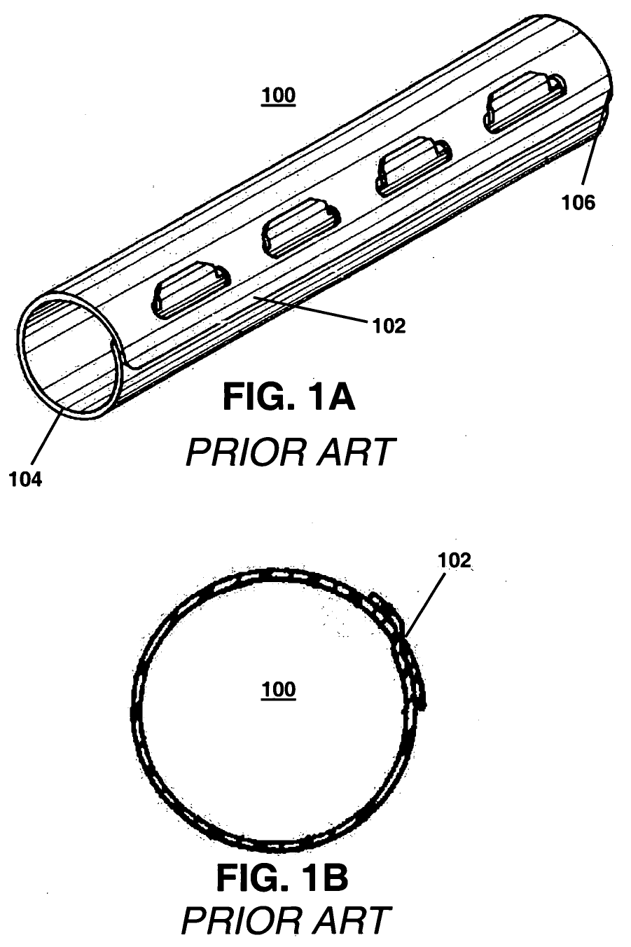

### DESCRIPTION OF THE PRESENTLY PREFERRED EMBODIMENTS

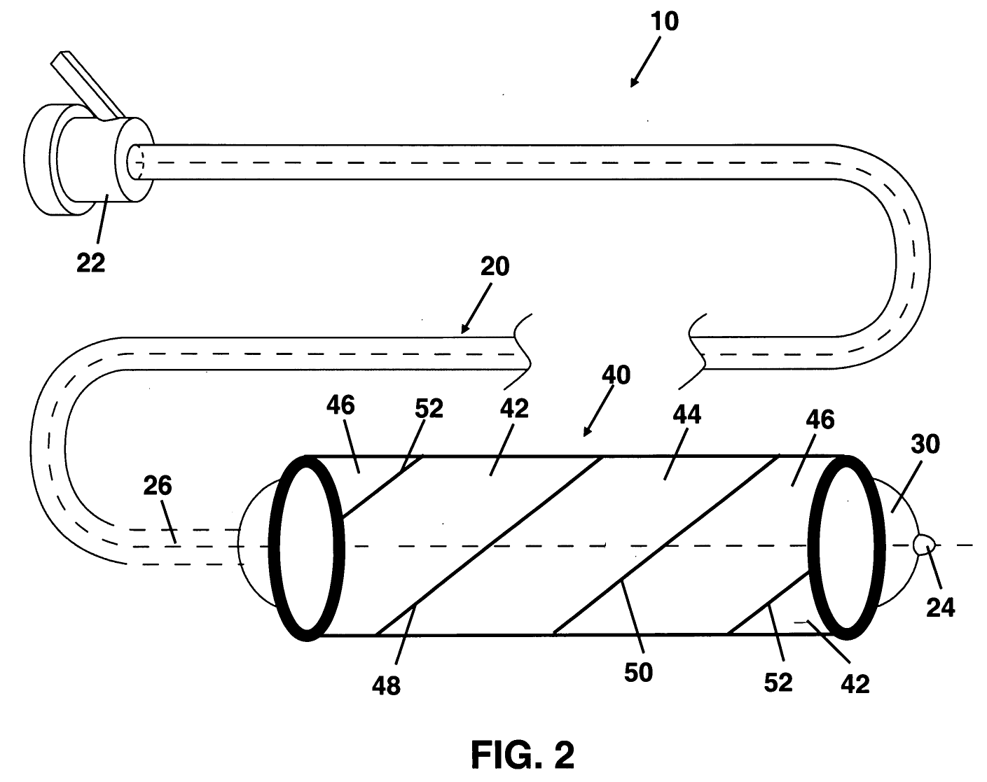

Referring to the drawings, which are not necessarily drawn to scale and wherein like reference numerals refer to like elements, FIG. 2 is a perspective view of an intraluminal stent delivery system in accordance with one embodiment of the present invention and shown generally by numeral 10. System 10 may include a catheter 20, a balloon 30 operably attached to the catheter 20, and a stent 40 disposed on the balloon 30. Stent 40 is shown in an assembled and compressed configuration in FIG. 2 and typically remains as such on the balloon 30 during advancement through the vasculature. The compressed stent 40 includes a relatively small profile (i.e., cross-sectional size) to minimize contact with surfaces, such as a vessel wall.

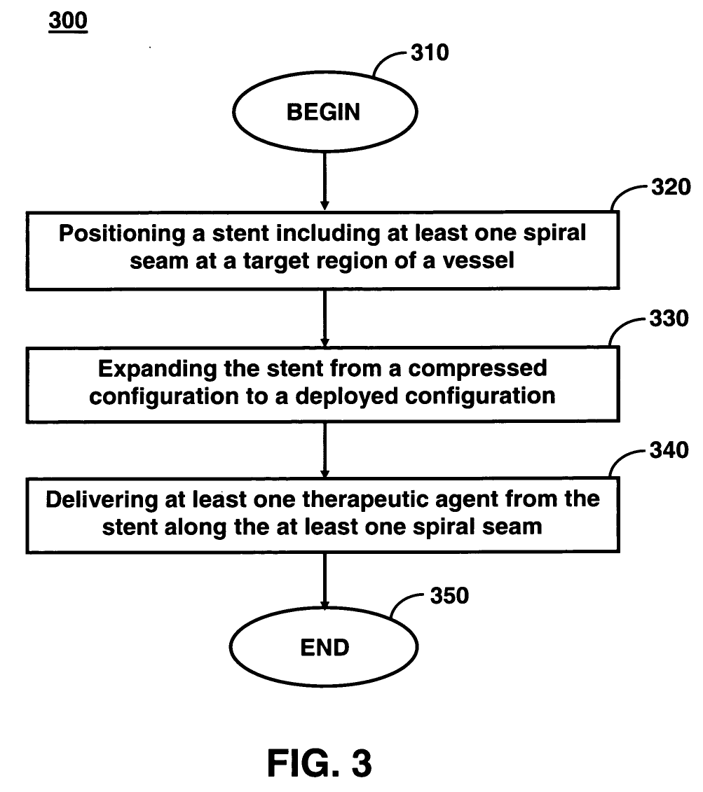

FIG. 3 illustrates a flowchart of a method of treating a vascular condition, in accordance with one embodiment of the present invention. The treatment of a vascular condition, such as a widening an obstructed blood vessel narrowed by plaque deposits, may begin at step 310. At step 320, the stent 40 is positioned at a target region of a vessel. Once the stent 40 is properly positioned within the target region, the stent 40 is expanded. In one embodiment, the stent 40 is expanded with the balloon 30. Balloon 30 may then be deflated and retracted thereby allowing the stent 40 to remain in a deployed configuration (step 330). The advancement, positioning, and deployment of stents and like devices are well known in the art. In addition, those skilled in the art will recognize that numerous devices and methodologies may be adapted for deploying the stent in accordance with the present invention. As discussed below, at least one therapeutic agent is delivered from the stent 40 along the at least one spiral seam 48, 50, and 52 (step 340). The treatment procedure may end at step 350.

The terms “catheter” and “stent”, as used herein, may include any number of intravascular and/or implantable prosthetic devices (e.g., a stent-graft); the examples provided herein are not intended to represent the entire myriad of devices that may be adapted for use with the present invention. Although the devices described herein are primarily done so in the context of deployment within a blood vessel, it should be appreciated that intravascular and/or implantable prosthetic devices in accordance with the present invention may be deployed in other vessels, such as a bile duct, intestinal tract, esophagus, and airway.

Catheter 20 may comprise an elongated tubular member manufactured from one or more polymeric materials, sometimes in combination with metallic reinforcement. In some applications (such as smaller, more tortuous arteries), it is desirable to construct the catheter from very flexible materials to facilitate advancement into intricate access locations. Numerous over-the-wire, rapid-exchange, and other catheter designs are known and may be adapted for use with the present invention. Catheter 20 may be secured at its proximal end to a suitable Luer fitting 22, and may include a distal rounded end 24 to reduce harmful contact with a vessel. Catheter 20 may be manufactured from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax® resin, Vestamid® nylon, Tecoflex® resin, Halar® resin, Hyflon® resin, Pellathane® resin, combinations thereof, and the like. Catheter 20 may include an aperture formed at the distal rounded end 24 allowing advancement over a guidewire 26.

Balloon 30 may be any variety of balloons or other devices capable of expanding the stent 40 (e.g., by providing outward radial forces). Balloon 30 may be manufactured from any sufficiently elastic material such as polyethylene, polyethylene terephthalate (PET), nylon, or the like. Those skilled in the art will recognize that the stent 40 may be expanded using a variety of means and that the present invention is not limited strictly to balloon expansion.

In one embodiment, the stent 40 may include a generally tubular unit defining a passageway extending along a longitudinal axis. Stent 40 is formed from at least one, and in this case three, stent units 42, 44, and 46 attached side-by-side. Spiral seams 48, 50, and 52 are provided between adjoining stent units (i.e., spiral seam 48 between stent unit 42, 44; spiral seam 50 between units 44, 46; and spiral seam 52 between units 42, 46). The overall length of the stent 40 is variable. In another embodiment, the stent 40 may be formed of any desired numbered of units, however, three to four units require less assembly while still providing a relative strong spiral seam.

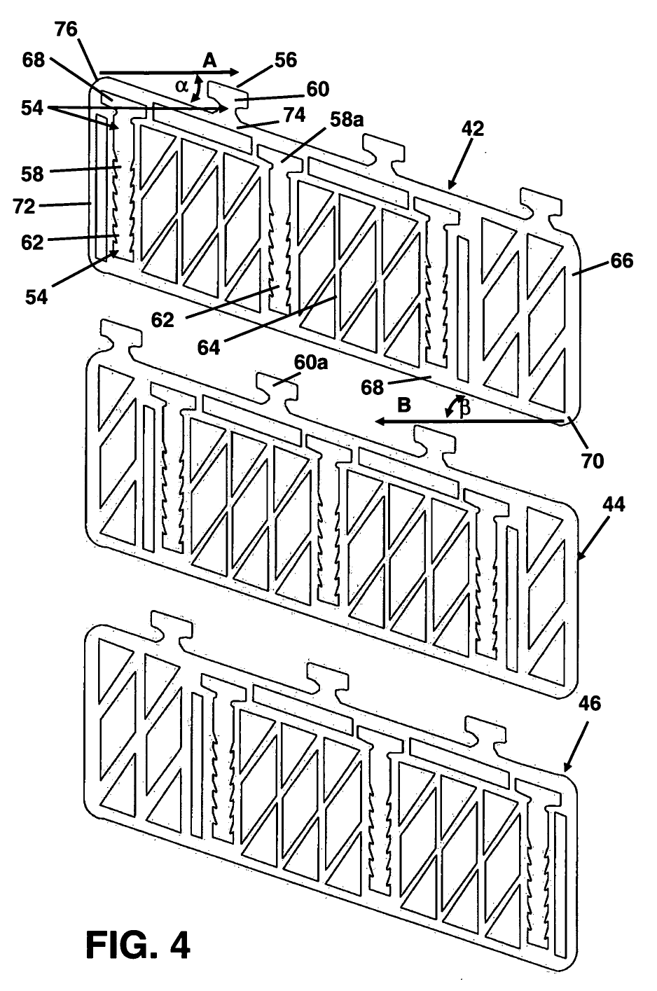

Referring to FIG. 4, the stent unit 42, which in this case is comparable to stent units 44, 46, may include, in one embodiment, a plurality of struts 64. Struts 64 are generally W-shaped in a repeating zig-zag configuration. In another embodiment, the struts 64 may be shaped and/or configured in a variety of different number, patterns, sizes, and configurations. Those skilled in the art will recognize that the number, pattern, size, and configuration of the struts 64 may vary from the illustrations and description provided herein. In another embodiment, the stent 60 may be solid, porous, or include other surface topographies.

Stent unit 42 includes a frame 66 surrounding the struts 64. Frame 66 is angled. Specifically, an angle, α, is formed between a frame side 74 and an axis A extending from a frame corner 76. Axis A is perpendicular to frame side 72. In one embodiment, the angle, α, may be about ten to twenty degrees. In another embodiment, the angle, α, may vary. The angle, α, is inversely proportional to the pitch of the spiral seam 48. As such, the overall length and strength of the spiral seam 48 increases with the angle, α. Spiral seam 48 provides a stronger seam compared to the seam 102 of the stent 100. Specifically, when stents are subjected to radial forces, the spiral seam 48 is less subject to developing localized weak points than the longitudinal seam 102. As used herein, the frame of stent 100 is not angled as any two sides that meet at a corner are perpendicular one to another. As such, the seam 102 is not a spiral.

In one embodiment, the frame 66 shape is that of a parallelogram (i.e., a quadrilateral with opposite sides parallel and, therefore, opposite angles equal). As any two opposing sides of the frame 66 are about parallel, the angle, α, is about equal to an angle, β, which is formed between a frame side 74 and axis B at frame corner 76. In another embodiment, the frame 66 may vary from the parallelogram shape, such as a trapezoid. Those skilled in the art will appreciate that the angles, α, β, may vary while still providing a spiral seam.

In one embodiment, a plurality of, and in this case three, lock assemblies 54 may be operably attached to the stent unit 42. The lock assembly 54 may be a ratchet assembly 56, as shown in FIG. 4, a hook, or other fastening means. Ratchet assembly 56 may include a lock portion 58 including an aperture 68 formed therein for receiving a tab portion 60. Lock portion 58 may include a plurality of teeth 62 for progressively engaging the tab portion 60. Ratchet assemblies 56 allow sliding of the stent unit 42 in a direction of deployment (i.e., increasing inner diameter of the stent 40) while also minimizing recoil in a direction of compression (i.e., decreasing inner diameter of the stent 40). In one embodiment, the tab portions 60 are positioned on the external surface of the stent 40 (i.e., in contact with the vessel wall). In such a position, the tab portions 60 may assist in anchoring the stent 40 within the vessel while providing a smooth interior surface that would not impede blood flow. Tab portions 60 positioned on the external surface of the stent 60 can create small local pockets where the stent 40 surface may not closely contact the vessel wall initially. However, such pockets would likely be filled in relatively quickly by cell migration as the stent 60 becomes encapsulated by the adjacent endothelial cells. In another embodiment, the tab portion 60 may be positioned flush with or inside the stent 40 surface.

In another embodiment, the number of lock assemblies may vary, for example, based on such factors as the length of the stent, the stent material properties, and the external stresses exerted on the stent.

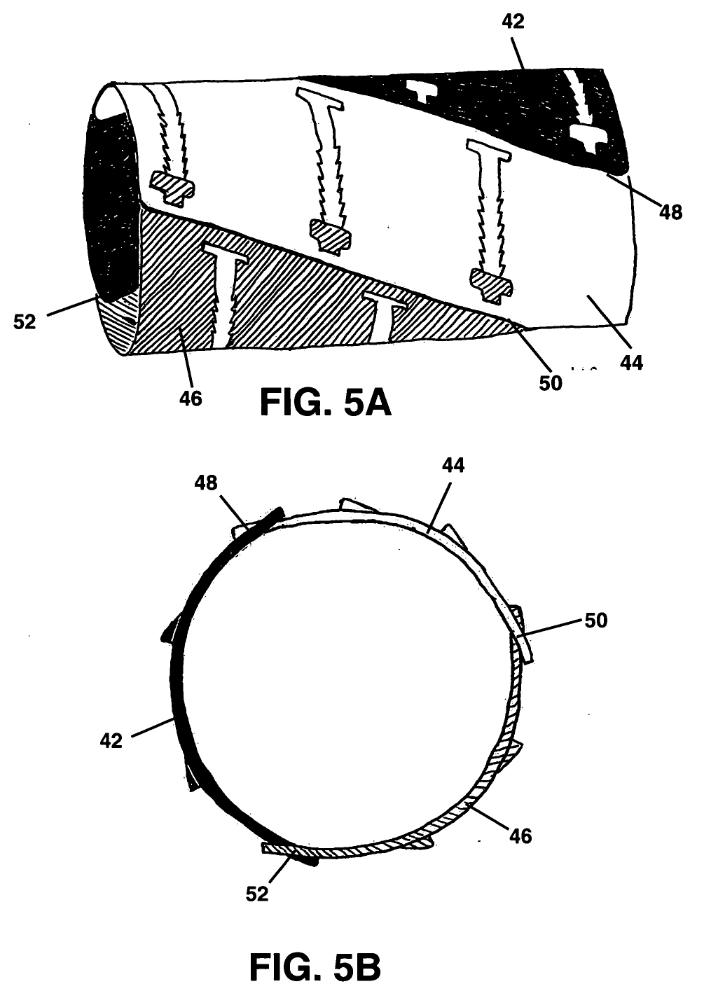

As shown in FIG. 4, for example, tab portion 60 of frame 42 corresponds with an aperture 72 of adjacent frame 46. Specifically, tab portion 60 is positioned into correspondingly shaped tab receiving opening 74 while in the compressed state. As the stent 60 is deployed, the tab portion 60 moves toward the teeth 62c of frame 42 thereby ratcheting the stent 60 into the deployed position, which is shown in FIGS. 5A and 5B.

In one embodiment, the ratchet assemblies 56 are positioned in a staggered configuration. Specifically, tab portions 60 of stent unit 42, are offset from (i.e., not parallel to) tab portions 60 of stent unit 44. Likewise, tab portions 60 of stent unit 44, are offset from (i.e., not parallel to) tab portions 60 of stent unit 46. The staggered configuration of the stent units 42, 44, and 46 allows minimal overlap of the ratchet assemblies 56 one to another when the stent 40 is compressed therefore providing a relatively small profile size. Those skilled in the art will recognize that the configuration of the lock assemblies (e.g., the lock assemblies 54) may vary from the staggered configuration.

In one embodiment, the tab portions 60 are positioned substantially planar to the frame 42, thereby minimizing crossing profile. Laser cutting of polymeric sheets is conducive to producing planar tab portions. In another embodiment, the tab portions 60 may not be planar thereby potentially providing a greater locking force. Molding of components may be more conducive to producing non-planar tab portions.

Once properly positioned within a vessel lumen, the balloon 30 and compressed stent 40 may be expanded together. Stent 40 may move radially outward from the longitudinal axis as the stent 40 expands. At least one (radiopaque) marker may be disposed on the stent 40, catheter 20, and or component thereof to allow in situ visualization and proper advancement, positioning, and deployment of the stent 40. The marker(s) may be manufactured from a number of materials used for visualization in the art including radiopaque materials platinum, gold, tungsten, metal, metal alloy, and the like. Marker(s) may be visualized by fluoroscopy, IVUS, and other methods known in the art. Those skilled in the art will recognize that numerous devices and methodologies may be utilized for deploying a stent and other intraluminal device in accordance with the present invention.

During deployment from the compressed configuration, for example, center tab portion 60a of stent unit 44 engages center lock portion 58a of stent unit 42. As stent 40, expands, center lock portion 58a progressively prevents stent units 42, 44 from re-compressing (i.e., recoil) due to a ratcheting action. At each point of expansion, the tab portion 60a passes across a tooth 62 of the center lock portion 58a at which there is a localized increase in of force on the spiral seam 98. However, a large force is placed on the spiral seam 98 during the lifetime of the stent 60. For example, if the stent 60 is deployed in a superficial femoral artery (SFA), there are many external forces applied as the patient walks, runs, or even holds an object in their lap. In a coronary application, contractions of the heart may impart forces on the stent 60 as well.

In one embodiment, the lock portion 58 is roughly parallel to frame side 72. This allows radial expansion of the stent 60 without an overall change in length. In addition, the lock portion 58 is preferably aligned with its corresponding tab portion 60a to allow easy insertion thereof.

Those skilled in the art will recognize that the structure of the ratchet may vary from the illustrated and described embodiment.

In one embodiment, the stent units 42, 44, and 46 may be manufactured from an inert, biocompatible material with high corrosion resistance. The biocompatible material should ideally be plastically deformed at low-moderate stress levels. In another embodiment, the stent 40 may be of the self-expanding variety and the stent units 42, 44, and 46 manufactured from, for example, a nickel titanium alloy and/or other alloy(s) that exhibit superelastic behavior (i.e., capable of significant distortion without plastic deformation). Other suitable materials for the stent 40 include, but are not limited to, ceramic, cobalt, tantalum, stainless steel, titanium ASTM F63-83 Grade 1, niobium, high carat gold K 19-22, MP35N, metals, metal alloys, and combinations thereof.

In one embodiment, the stent units 42, 44, and 46 may be manufactured by a thermal pressing, injection molding, or other process known in the art. In another embodiment, the stent units 42, 44, and 46 may be formed by laser cutting a biodegradable polymer film into a finished form, shown in FIGS. 5A and 5B. In one embodiment, the stent units 42, 44, and 46 may be assembled by inserting the ratchet assemblies one to another forming the cylindrical stent 40. The stent 40 may them be loaded onto the balloon 30 and compressed as known in the art for subsequent deployment.

Stent 40 may include at least one therapeutic agent 80 as part of one or more coatings. Application of the therapeutic agent 80 may be performed at numerous points during stent 40 manufacture (e.g. before laser cutting, after compression onto the balloon 30, etc.). The coatings may be positioned on various portions of the stent 40. In one embodiment, the therapeutic agent 80 may comprise one or more drugs, polymers, a component thereof, a combination thereof, and the like. For example, the therapeutic agent may include a mixture of a drug and a polymer as known in the art. Some exemplary drug classes that may be included are antiangiogenesis agents, antiendothelin agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, and the like. Specific example of drugs include abciximab, angiopeptin, colchicine, eptifibatide, heparin, hirudin, lovastatin, methotrexate, sirolimus, zotarolimus, streptokinase, taxol, ticlopidine, tissue plasminogen activator, trapidil, urokinase, and growth factors VEGF, TGF-beta, IGF, PDGF, and FGF.

The polymer generally provides a matrix for incorporating the drug within the coating, or may provide means for slowing the elution of an underlying therapeutic agent when it comprises a cap coat. Some exemplary biodegradable polymers that may be adapted for use with the present invention include, but are not limited to, polycaprolactone, polylactide, polyglycolide, polyorthoesters, polyanhydrides, poly(amides), poly(alkyl-2-cyanocrylates), poly(dihydropyrans), poly(acetals), poly(phosphazenes), poly(dioxinones), trimethylene carbonate, polyhydroxybutyrate, polyhydroxyvalerate, their copolymers, blends, and copolymers blends, combinations thereof, and the like. Exemplary non-biodegradable polymers that may be adapted for use with the present invention may be divided into at least two classes. The first class includes hydrophobic polymers such as polyolefins, acrylate polymers, vinyl polymers, styrene polymers, polyurethanes, polyesters, epoxy, nature polymers, their copolymers, blends, and copolymer blends, combinations thereof, and the like. The second class includes hydrophilic polymers, or hydrogels, such as polyacrylic acid, polyvinyl alcohol, poly(N-vinylpyrrolidone), poly(hydroxy-alkylmethacrylate), polyethylene oxide, their copolymers, blends and copolymer blends, combinations of the above, and the like.

Solvents are typically used to dissolve the therapeutic agent and polymer to comprise a therapeutic agent coating solution. Some exemplary solvents that may be adapted for use with the present invention include, but are not limited to, acetone, ethyl acetate, tetrahydrofuran (THF), chloroform, N-methylpyrrolidone (NMP), methylene chloride, and the like.

Those skilled in the art will recognize that the nature of the drug and polymer may vary greatly and are typically formulated to achieve a given therapeutic effect, such as limiting restenosis, thrombus formation, hyperplasia, etc. Once formulated, a therapeutic agent solution (mixture) comprising the coating may be applied to the stent 40 by any of numerous strategies known in the art including, but not limited to, spraying, dipping, rolling, nozzle injection, and the like. Numerous strategies of applying the coating in accordance with the present invention are known in the art.

Therapeutic agent 80 may accumulate within the spiral seam 48, similar in fashion as with the prior art stents within the seams 102. However, as the spiral seam 48 rotates about the stent 40 body (rather than running linearly along the stent 100), it is capable of delivering the therapeutic agent 80 in a more homogeneous fashion.

While the embodiments of the invention disclosed herein are presently considered to be preferred, various changes and modifications can be made without departing from the spirit and scope of the invention. For example, the number of stent units (e.g., single or multiple unit designs), angle, lock assemblies, struts, and frame are not limited to the illustrated and described embodiments.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. An intraluminal stent comprising a framework including an elongated spiral seam, the framework comprising a side edge including an angle formed with respect to a frame corner.

2. The stent of claim 1 wherein the stent comprises at least one stent unit, each unit including a portion of the spiral seam.

3. The stent of claim 1 wherein the angle is about ten to twenty degrees.

4. The stent of claim 1 wherein the stent comprises a biodegradable polymer film.

5. The stent of claim 1 further comprising a plurality of lock assemblies operably attached to the stent.

6. The stent of claim 5 wherein the plurality of lock assemblies comprise ratchet assemblies for allowing sliding of the stent unit in a direction of deployment and minimizing recoil in a direction of compression.

7. The stent of claim 5 wherein the plurality of lock assemblies comprise a staggered configuration.

8. The stent of claim 1 further comprising at least one therapeutic agent disposed on the stent.

9. An intraluminal stent delivery system comprising:
a catheter; and
an intraluminal stent comprising a framework including an elongated spiral seam, the framework comprising a side edge including an angle formed with respect to a frame corner.

10. The system of claim 9 wherein the stent comprises at least one stent unit, each unit including a portion of the spiral seam.

11. The system of claim 10 wherein the angle is about ten to twenty degrees.

12. The system of claim 9 wherein the stent comprises a biodegradable polymer film.

13. The system of claim 9 further comprising a plurality of lock assemblies operably attached to the stent.

14. The system of claim 13 wherein the plurality of lock assemblies comprise ratchet assemblies for allowing sliding of the stent unit in a direction of deployment and minimizing recoil in a direction of compression.

15. The system of claim 13 wherein the plurality of lock assemblies comprise a staggered configuration.

16. The system of claim 9 further comprising at least one therapeutic agent disposed on the stent.

17. A method of treating a vascular condition, the method comprising:
positioning a stent including at least one spiral seam at a target region of a vessel;
expanding the stent from a compressed configuration to a deployed configuration; and
delivering at least one therapeutic agent from the stent along the at least one spiral seam.

18. The method of claim 17 wherein the stent material comprises a biodegradable polymer film.

19. The method of claim 17 wherein expanding the stent from a compressed configuration to a deployed configuration comprises allowing sliding of the stent in a direction of deployment and minimizing recoil in a direction of compression.

20. The method of claim 17 wherein the spiral seam is angled at about 10 to 20 degrees.
